(19) United States
(12) Patent Application Publication
(10) Pub. No.: US 2010/0049181 A1
(43) Pub. Date: Feb. 25, 2010

(54) MEDICAL LIGHT DEVICE

(75) Inventors: HUNG CHIN LIN, HSINCHU COUNTY (TW); CHIA CHEN CHANG, HSINCHU COUNTY (TW)

Correspondence Address: WPAT, PC;INTELLECTUAL PROPERTY ATTORNEYS, 2030 MAIN STREET, SUITE 1300, IRVINE, CA, 92614, US

(73) Assignee: ADVANCED OPTOELECTRONIC TECHNOLOGY INC., HSINCHU COUNTY (TW)

(21) Appl. No.: 12/540,097

(22) Filed: Aug. 12, 2009

(30) Foreign Application Priority Data
Aug. 19, 2008 (TW) 097131524

(51) Int. Cl. A61B 18/18; A61N 5/06

(52) U.S. Cl. 606/13; 607/90

(57) ABSTRACT

A medical light device includes a main body, a light source, a filler, and a contact part. A cavity, receiving a light source, is disposed on the main body. The filler is composed of transparent material, filled inside the cavity, and covers the light source. The contact part, composed of soft and transparent material, is placed adjacent to the filler, covering the surface of the filler. The refractive index difference between the contact part and the filler is smaller than the refractive index difference between the filler and air.

### BACKGROUND OF THE INVENTION

1. Field of the Invention

The present invention relates to a medical light device, and relates more particularly to a medical light device having a contact part.

2. Description of the Related Art

In the medical field, there are more and more opportunities for treatment with light therapy. Currently, there are three well-known types of light therapy. The first type is laser therapy, which involves directing a laser beam to the affected area of a patient and is generally used in treating cancer diseases, removing skin marks or spots, or plastic surgery. However, the drawback of laser therapy is that the laser beam can cause damage to normal cells near the affected area.

The second type of light therapy is photodynamic therapy. Photodynamic therapy involves initially applying a photosensitizer to a patient's skin, and waiting while the skin surface absorbs the photosensitizer. Then a suitable light is radiated on the skin surface to destroy malignant cells by photochemical reaction. In photodynamic therapy, the light can be emitted from, for example, a light emitting diode (LED). Abnormal epidermal cells, hair follicles, and particularly the sebaceous gland can easily absorb the photosensitizer, and during the process of the therapy no heat is generated so the photodynamic therapy can be used not only in skin cancer treatment, but also in wrinkle removal, acne treatment, and hair removal.

The third type of light therapy is low energy photomodulation therapy, which uses low energy light emitted from a light emitting diode and in a specific waveband to stimulate fibroblastic cells and to modulate the cells' cytochrome to produce collagen and elastin which aging skin has lost and needs. During the therapy no heat is generated due to photomodulation effect that is used in wrinkle removal and in anti-aging skin treatment.

During the above-mentioned photodynamic therapy and low energy photomodulation therapy, one method of irradiating the affected area of the patient involves the use a light pen. FIG. 1A shows a traditional light pen being used for performing therapy. FIG. 1B is a cross sectional view of a traditional light pen. Referring to FIGS. 1A and 1B, the light pen 100 includes a handle portion 110, a light emitting diode 120 and a filler 130, wherein the handle portion 110 has a cavity 112 and the light emitting diode 120 is placed on the bottom surface of the cavity 112. In addition, the filler 130 stuffed inside the cavity 112 is made of rubber for medical use. During treatment, a user grasps the handle portion 110 of the light pen 100, with the light emitting diode turned on. The light emitting diode emitting light in a specific waveband radiates the affected area 4 of a patient to perform therapy.

Referring to FIG. 1B, because the light refraction index of the filler 130 is greater than that of air, a light beam I₁ from the light emitting diode 120 is refracted away from the normal when it passes the interface between the filler 130 and air. As a result, the light beam I₁ cannot reach the affected area 4, but radiates on normal cells so that light energy is wasted.

Thus, there is a need for persons skilled in the art to investigate a means to direct the light beam I₁ from the light emitting diode 120 to efficiently radiate the affected area 4 of a patient.

### SUMMARY OF THE INVENTION

The objective of the present invention is to provide a medical light device, which can radiate light more efficiently onto the affected area of a patient.

In accordance with the above objective, the present invention proposes a medical light device, which comprises a main body, a light source, a filler, and a contact part. The main body includes a cavity, which receives the light source. In addition, the filler, composed of transparent material, is filled inside the cavity and covers the light source. Moreover, the contact part is placed adjacent to the filler and covers the surface of the filler. The contact part is composed of a transparent and soft material, wherein the refractive index difference between the contact part and the filler is smaller than the refractive index difference between the filler and air.

In the above-mentioned medical light device, a first connection member is disposed on the filler and a second connection member is disposed on the contact part, wherein the first connection member is able to engage to the second connection member. Specifically, the second connection member has a head portion larger than its neck portion, and the first connection member has a shape correspondingly matched to the second connection member.

In the above-mentioned medical light device, the material of the contact part is silicone for medical use.

In the above-mentioned medical light device, the light source is a light emitting diode array.

In the above-mentioned medical light device, the refraction index of the contact part is the same as the refraction index of the filler.

In the above-mentioned medical light device, the main body has a stick-like shape.

Because the refractive index difference between the contact part and the filler is smaller than the refractive index difference between the filler and air, light passing the interface between the contact part and the filler refracts less than light passing the interface between the filler and air. The medical device wastes less light energy than the traditional light pen.

To better understand the above-described objectives, characteristics and advantages of the present invention, embodiments, with reference to the drawings, are provided for detailed explanations.

### BRIEF DESCRIPTION OF THE DRAWINGS

The invention will be described according to the appended drawings in which:

FIG. 1A shows a traditional light pen, which is being used for performing therapy;

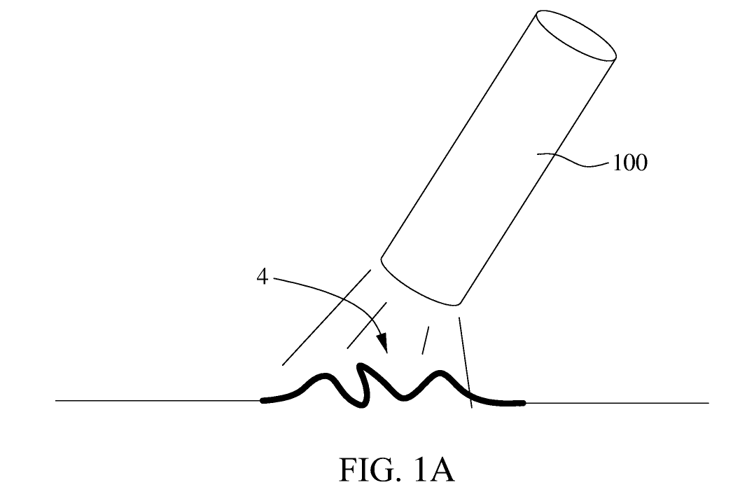

FIG. 1B is a partial cross sectional view of a traditional light pen;

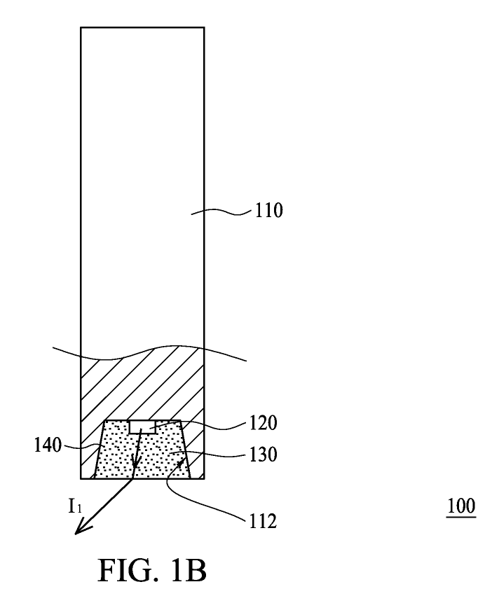

FIG. 2A is a perspective view of a medical light device, which is being used for a treatment, according to one embodiment of the present invention;

FIG. 2B is a partial cross-sectional view of a medical light device according to one embodiment of the present invention; and

FIG. 3 shows the connection between the first connection member of the filler and the second connection member of the contact part according to one embodiment of the present invention.

### DETAILED DESCRIPTION OF THE INVENTION

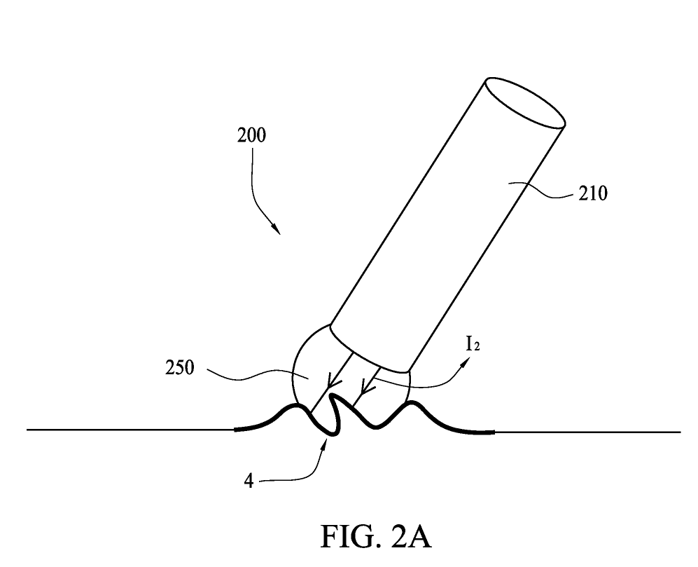

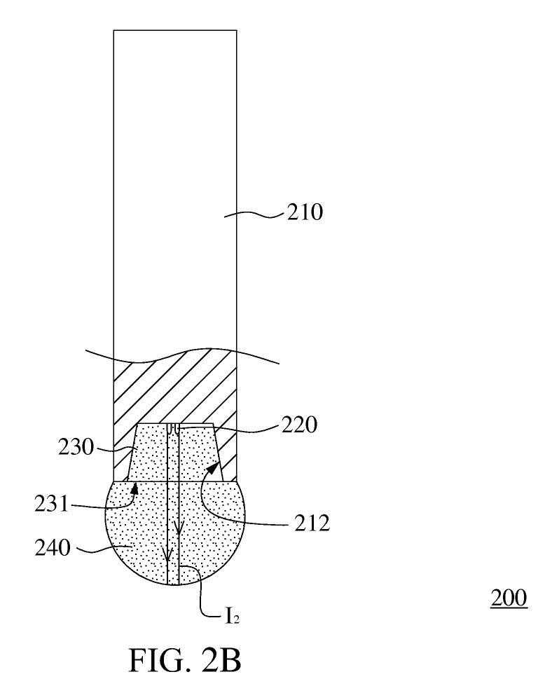

FIG. 2A is a perspective view of a medical light device which is being used for a treatment according to one embodiment of the present invention, and FIG. 2B is a partial cross-sectional view of a medical light device according to one embodiment of the present invention. Referring to FIGS. 2A and 2B, in the present embodiment, the medical device 200 comprises a main body 210, a light source 220, a filler 230, and a contact part 240. The main body 210 includes a cavity 212 receiving the light source 220. The light source 220 can be, for example, a light emitting diode array (LED array). The LED array comprises a plurality of light emitting diodes. In the present embodiment, the main body 210 can have a stick-like shape for convenience in grasping. In addition, persons skilled in the art know that the main body 210 can be designed to have other shapes.

The filler 230 is composed of transparent material, and in the present embodiment, the filler is made of rubber for medical use. The filler 230 is filled inside the cavity 212, covering the light source 220. In the present embodiment, the contact part 240 has a spherical shape. However, persons skilled in the art know that the contact part 240 can be designed to have other shapes, for example an elliptical shape. The contact part 240 is placed adjacent to the filler 230, covering the surface 231 of the filler 230. The contact part 240, composed of a transparent and soft material such as silicone for medical use, is configured to contact the affected area 4 of a patient.

In the present embodiment, the material of the contact part 240 and the filler 230 can be silicone for medical use so that both have the same refraction index. Therefore, the light beam I2 emitted from the light source 220 can pass linearly through the contact part 240 and the filler 230 to the affected area 4, and will not refract away from the normal. Consequently, in contrast to the traditional light pen 100, the medical device 200 of the present embodiment does not waste light energy. It is believed that the contact part 240 and the filler 230 having the same refraction index is preferred. However, persons skilled in the art know that to meet different application requirements, the contact part 240 and the filler 230 can have different refraction indexes. In fact, the refractive index difference between the contact part 240 and the filler 230 is smaller than the refractive index difference between the filler 230 and air, whereby the medical device 200 saves more light than the traditional light pen 100.

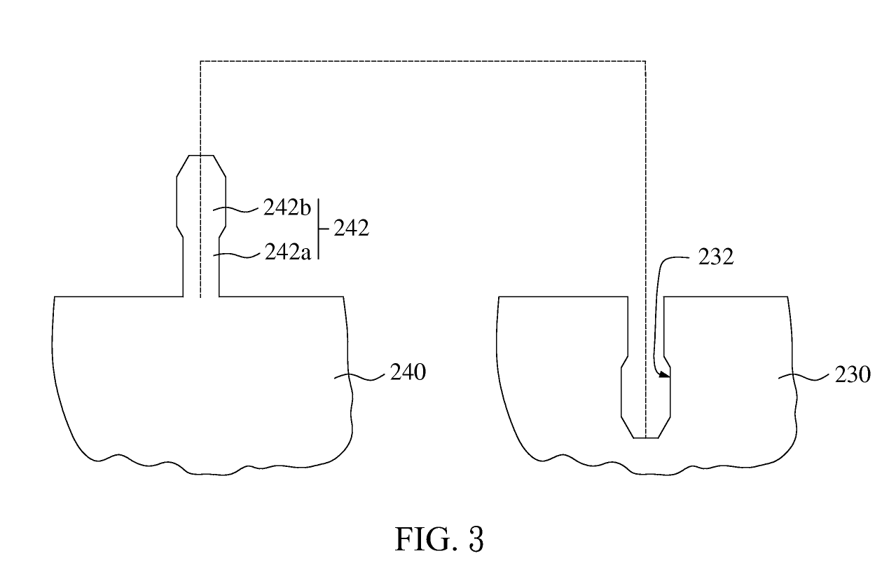

In the present embodiment, the contact part 240 can be detached from the filler 230. As such, when treatment is finished, the used contact part 240 can be removed and replaced with a new contact part 240 to avoid cross infection. The contact part 240 and the filler 230 can be adhered together using adhesive means, or can be assembled by a connecting mechanism. FIG. 3 shows the connection between the first connection member 232 of the filler 230 and the second connection member 242 of the contact part 240 according to one embodiment of the present invention. The first connection member 232 disposed on the filler 230 is an indentation, which is correspondingly shaped to match and engage the second connection member 242 of the contact part 240. The second connection member 242 is a protrusion, which has a head portion larger than its neck portion. The second connection member 242 comprises a connecting portion 242a and a lock portion 242b. The connecting portion 242a connects the second connection member 242 to the lock portion 242b, wherein the average width of the connecting portion 242a in a radial direction is less than that of the lock portion 242b. By the engagement between the first connection member 232 and the second connection member 242, the contact part 240 can be attached to the filler 230. It is known that the above-mentioned first connection member 232 and second connection member 242 just show an example. In fact, in the present invention, the shape of the first connection member 232 and the second connection member 242 are not limited to the embodiment. Any shape that can connect the contact part 240 to the filler 230 is included in the present invention. For example, persons skilled in the art can also design the first connection member 232 as a head portion larger than its neck portion, and design the second connection member 242 as an indentation.

The above-described embodiments of the present invention are intended to be illustrative only. Numerous alternative embodiments may be devised by persons skilled in the art without departing from the scope of the following claims.

What is claimed is:

1. A medical light device, comprising:
a main body having a cavity;
a light source disposed within said cavity;
a filler composed of transparent material, said filler filled inside said cavity and covering said light source; and
a contact part adjacent to said filler and covering the surface of said filler, said contact part composed of transparent and soft material, wherein the refractive index difference between said contact part and said filler is smaller than the refractive index difference between said filler and air.

2. The medical light device of claim 1, further comprising a first connection member disposed on said filler and a second connection member disposed on said contact part, wherein said first connection member is able to engage to said second connection member.

3. The medical light device of claim 2, wherein said second connection member is a taper protrusion with a smaller base, and said first connection member is an indentation for engagement with the profile of the second connection member.

4. The medical light device of claim 1, wherein the material of said contact part is silicone for medical use.

5. The medical light device of claim 1, wherein said light source is a light emitting diode array.

6. The medical light device of claim 1, wherein the refraction index of said contact part is the same as the refraction index of said filler.

7. The medical light device of claim 1, wherein said contact part has a spherical shape.

8. The medical light device of claim 1, wherein said main body has a stick-like shape.
